(19) United States
(12) Patent Application Publication
(10) Pub. No.: US 2012/0109332 A1
(43) Pub. Date: May 3, 2012

(54) MEDICAL DEVICE FOR RELEASING IN A HOLLOW ORGAN AND INSERTION SYSTEM FOR MEDICAL DEVICES

(75) Inventors: Werner Mailaender, Engelsbrand (DE); Giorgio Cattaneo, Karlsruhe (DE)

(73) Assignee: ACANDIS GMBH & CO. KG, Pfinztal (DE)

(21) Appl. No.: 13/320,148

(22) PCT Filed: May 12, 2010
(86) PCT No.: PCT/EP2010/002937

§ 371 Date: Dec. 1, 2011

(30) Foreign Application Priority Data
May 13, 2009 (DE) 10 2009 021 039.3

(51) Int. Cl. A61F 2/04

(52) U.S. Cl. 623/23.7

(57) ABSTRACT

A medical device for releasing in a hollow organ having a hollow cylindrical body that can be transferred in a compressed state having a reduced diameter and in an expanded state having an enlarged diameter. The body having a stop means at the proximal and/or distal axial end thereof having at least one retaining element connected to the body by a connecting segment. The retaining element and the body are decoupled by the connecting segment such that the retaining element has a smaller curvature in the compressed state than the body. The connecting segment elastically connects the retaining element and the body, wherein the contour of the retaining element in the compressed state of the body protrudes past the outer circumference of the body such that the retaining element is displaceable radially inward relative to the body into a stop position through the inner wall of an insertion system.

The invention relates to a medical device for releasing in a hollow organ and to an insertion system for medical devices.

A device for releasing in a hollow organ, having the features of the preamble of claim 1, and an insertion system are known from EP 1 157 673 A2, for example, which discloses a stent for transluminal implantation in hollow organs and also an insertion catheter for such a stent. For implantation, the stent located in the compressed state in the insertion catheter is advanced to the location to be treated in the hollow organ and is positioned there. To release the stent, the outer sheath of the insertion catheter is drawn back, such that the stent expands and supports the hollow organ.

In the event that the position of the stent released from the insertion catheter has to be corrected, the stent should be able to be repositioned by the insertion catheter. For this purpose, the stent is usually fixed axially on a guide wire of the insertion catheter, such that the stent can be drawn back together with the guide wire into the insertion catheter. In the stent system according to EP 1 157 673 A2, the ability to reposition the stent is afforded by a positioning element that is secured on the guide wire or mandrel of the insertion catheter. The positioning element has several recesses which are distributed on the circumference and into which correspondingly shaped retaining segments at the axial end of the stent engage with a form fit. By means of the form-fit connection between the positioning element and the stent, a locking effect is provided that acts in the axial direction and permits a repositioning of the stent.

In the field of interventional neurology, particularly in the field of stent and catheter design, the trend in development work is toward miniaturization. If the repositioning functionality described above is to be incorporated into the design, the limits of what is possible and of what can be produced are soon reached. This also applies to the stent system that is known from EP 1 157 673 A2 since, for reasons of strength, the positioning element must have sufficient thickness in the wall area between the recesses, and this makes miniaturization difficult or prevents it.

A further stent system is known from U.S. Pat. No. 7,303,580 B2, in which the stent is fixed on the guide wire by eyelets and by rivets engaging through the latter, which protrude into the lumen of the stent and engage behind a limit stop on the guide wire. This kind of fixing arrangement cannot be made smaller and also has the disadvantage that, in the implanted state, the eyelets protrude into the lumen of the stent and thus impede the flow of blood.

The object of the invention is to make available a medical device for releasing in a hollow organ, which device satisfies the condition for an axial locking effect in an insertion system for positioning or repositioning the device, wherein the device is well suited to a small construction. It is also the object of the invention to make available an insertion system for medical appliances, which insertion system has a device for releasing in a hollow organ, and which insertion system can be easily miniaturized.

According to the invention, the object is achieved, in respect of the medical device, by the subject matter of claim 1 and, in respect of the insertion system, by the subject matter of claim 9.

The invention is based on a medical device for releasing in a hollow organ, with a hollow cylindrical body that can be converted to a compressed state having a reduced diameter and to an expanded state having an enlarged diameter, the body having a locking means at its proximal and/or distal axial end. The locking means comprises at least one retaining element connected to the body by a connecting segment. The retaining element and the body are decoupled by the connecting segment in such a way that the retaining element has a smaller curvature than the body in the compressed state, in particular in the compressed state of the body, or generally in the compressed state of the device. Accordingly, the radius of curvature of the retaining element is greater than the radius of curvature of the body in the compressed state. The retaining element is less strongly curved than the body. The connecting segment connects the retaining element and the body elastically. The contour of the retaining element in the compressed state of the body protrudes beyond the outer circumference of the body in such a way that the retaining element can be moved radially inward relative to the body into a locking position by the inner wall of an insertion system.

The invention is moreover based on an insertion system for medical appliances having a device for releasing in a hollow organ, with a hollow cylindrical body that can be converted to a compressed state having a reduced diameter and to an expanded state having an enlarged diameter, the body having a locking means at its proximal and/or distal axial end. The locking means comprises at least one retaining element connected elastically to the body by a connecting segment. The retaining element and the body are decoupled by the connecting segment in such a way that the retaining element has a smaller curvature than the body in the compressed state, in particular in the compressed state of the body, or generally in the compressed state of the device. The contour of the retaining element touches the inner wall of the insertion system at at least one location, in such a way that that the retaining element is moved radially inward relative to the body into a locking position and cooperates with an element that is arranged in the insertion system and that fixes the body.

Thus, in the compressed state of the device, the retaining element is less curved, that is to say flatter, than the body. The compression of the device takes place substantially, in particular completely, in the form of a compression of the body. The retaining element is decoupled from the body, such that the compression of the body does not substantially lead to a change in shape of the retaining element.

In the context of the invention, the medical device for releasing in a hollow organ is claimed itself, that is to say independently of the insertion system, and an insertion system comprising such a medical device is also claimed.

The invention has the advantage that the medical device requires minimal space in the insertion system. In addition, the invention can be very easily produced since no additional positioning elements are needed.

In a preferred embodiment of the invention, one to four retaining elements are distributed on the circumference. This has the effect that the retaining elements can be formed with a sufficiently large circumferential extent such that the greatest possible inwardly directed radial movement of the retaining elements can be achieved. The retaining elements can be spaced apart on the circumference in the compressed state of the body. The free space between the retaining elements in the compressed state of the body avoids the retaining elements colliding with each other during the movement into the locking position.

In the compressed state of the body, the extent L₂ of a retaining element transverse to the longitudinal direction of the body can measure at least 5% of the total outer circumference of the body, in particular at least 7%, at least 10%, at least 12%, at least 14%, at least 16%, at least 18%, at least 20%, at least 22%, at least 24%, at least 26%, at least 28%, at least 30%, of the total outer circumference of the body.

The abovementioned lower limits for the transverse extent of a retaining element have proven expedient in ensuring a sufficiently great inward radial movement of the retaining elements. The radial movement increases as the size of the retaining elements increases.

Preferably, in the compressed state of the body, the extent L₂ of a retaining element transverse to the longitudinal direction of the body measures at most 32% of the total outer circumference of the body (10), in particular at most 30%, at most 28%, at most 26%, at most 24%, at most 22%, at most 20%, of the total outer circumference of the body. This creates different upper limits that are adapted to a different number of retaining elements.

A retaining element can be substantially circular. The circular shape of the retaining elements is particularly easy to produce.

In another embodiment, the retaining element comprises an X-ray marker and thus performs a dual function, that is to say, on the one hand, the locking function and, on the other hand, the detectability of the medical device.

The connecting segment can comprise at least one web arranged in the longitudinal direction of the body. The web provides a possibility of producing the elastic connection between the retaining element and the body. In the insertion system, provision can be made that the element arranged in the insertion system comprises a guide wire having at least one projection, wherein the retaining element engages behind the projection in the locking position. This embodiment is particularly easy to produce, since the projection is not subject to any particular geometric requirements.

In another embodiment, the guide wire has at least two projections between which the retaining element engages in the locking position. This has the effect that the retaining element and thus the medical device is fixed in both axial directions, such that the device can be moved in both axial directions by actuation of the guide wire.

The invention is explained in more detail below on the basis of illustrative embodiments and with reference to the attached schematic drawings, in which:

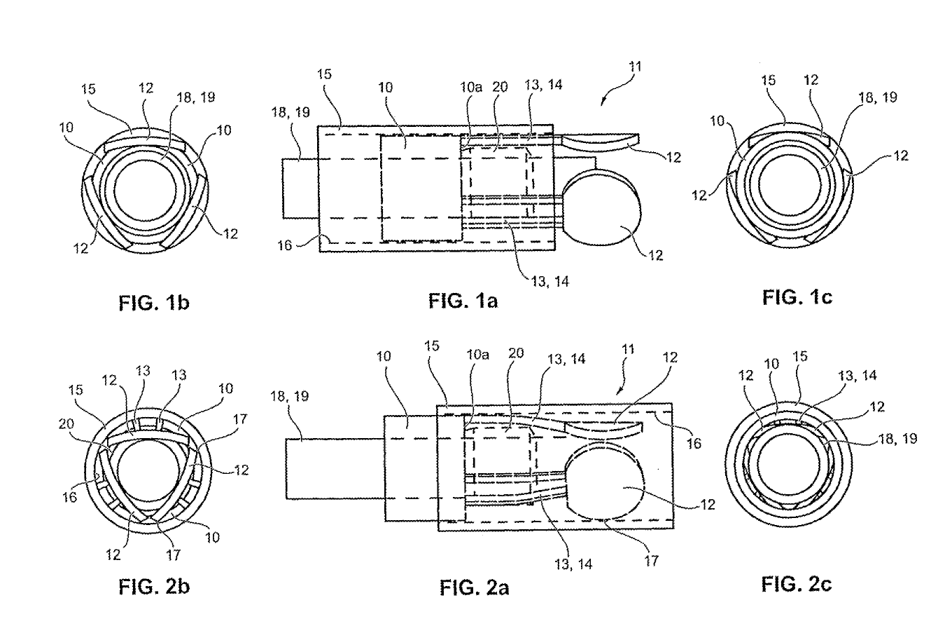

FIG. 1a shows a perspective side view of an insertion system with a stent and a guide wire, wherein the retaining elements of the stent are located outside the insertion system;

FIG. 1b shows a view of the system according to FIG. 1a in the distal direction;

FIG. 1c shows a view of the system according to FIG. 1a in the proximal direction;

FIG. 2a shows the system according to FIG. 1a, wherein the retaining elements are drawn into the insertion system;

FIG. 2b shows a view of the system according to FIG. 2a in the distal direction;

FIG. 2c shows a view of the system according to FIG. 2a in the proximal direction;

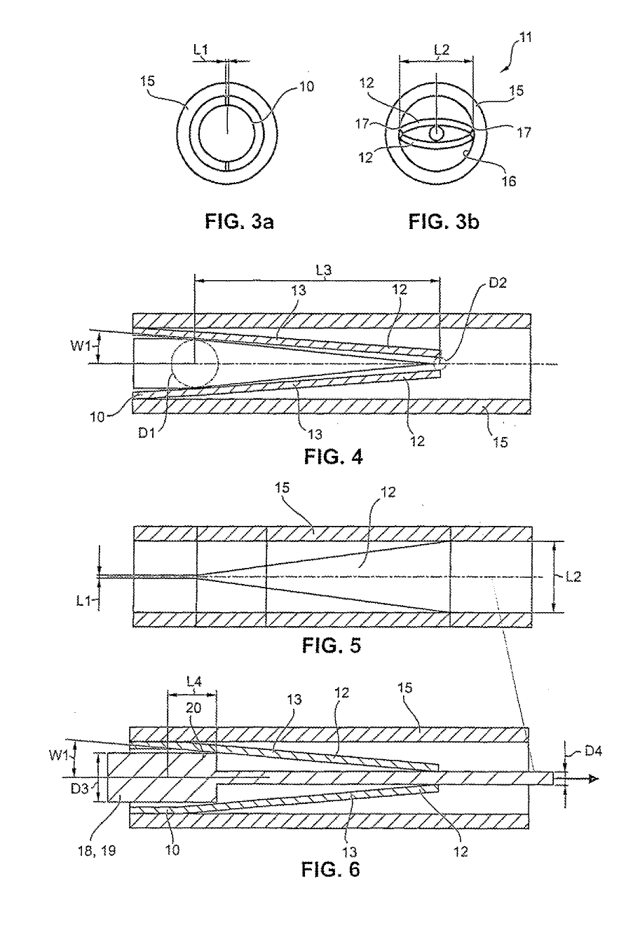

FIG. 3a shows a schematic cross section through an insertion system according to another illustrative embodiment, wherein the section runs between the retaining elements and the body;

FIG. 3b shows a cross section through the system according to FIG. 3a, wherein the section runs through the retaining elements;

FIG. 4 shows a longitudinal section through the system according to FIGS. 3a and 3b;

FIG. 5 shows a schematic plan view of the system according to FIG. 4; and

FIG. 6 shows a longitudinal section through the system according to FIG. 4 with a guide wire.

FIGS. 1a, 1b and 1c show an insertion system 15 for medical appliances having a device for releasing in a hollow organ. In the context of the invention, protection is claimed both for the medical device itself, that is to say without the insertion system, and also for the combination of the insertion system with the medical device. The medical device can be a stent, in particular for interventional neurology. The invention is not limited to stents and instead can be used generally for medical implants that require repositioning during implantation. The invention is also applicable to medical appliances, for example clot retrievers, that are not implanted but are temporarily released in the body. The invention is thus universally applicable in the field of medical technology and, by virtue of its simple design, has great economic potential.

All of the features disclosed below in connection with the stent according to FIG. 1a are disclosed in general form in connection with the medical device for releasing in a hollow organ.

As can be seen in FIG. 1, the stent has a body 10 with a distal axial end and a proximal axial end. FIG. 1a shows the proximal axial end 10a, which has a locking means 11. The locking means 11 can alternatively be arranged at the distal axial end (not shown). It is also possible for the locking means 11 to be provided at the distal and the proximal axial end. The locking means 11 at the distal and at the proximal axial end can be of an identical or similar design. The embodiment with a proximally arranged locking means 11 is particularly advantageous for releasing the stent, but the invention is not limited to this.

The locking means 11 comprises at least one retaining element 12, which is connected to the body 10 by a connecting segment 13. The retaining element 12 and the connecting segment 13 together form a flexible wing or a tongue, of which the function is to fix the stent axially in the insertion system 15. In the illustrative embodiment according to FIG. 1a, the retaining elements are each substantially circular or disk-shaped. It is also possible for the retaining elements 12 to be oval, for example, or to be designed with other geometries, for example in the form of elongate tongues that narrow in the direction of the body 10. In the present illustrative embodiment, three retaining elements 12 are distributed on the circumference. It is also possible for the locking function to be achieved using a single retaining element 12 or two retaining elements 12. It has proven expedient if up to four retaining elements 12 are distributed on the circumference. With a suitable geometric configuration of the retaining elements 12, it is also possible for more than four retaining elements 12 to be provided, for example five or six retaining elements 12, provided that the self-locking function of the retaining elements 12 in cooperation with the insertion system 15 is ensured.

FIG. 1a shows that the retaining elements 12 in the expanded state are spaced apart on the circumference. A collision of the retaining elements 12 during the movement into the locking position (FIG. 2a) is avoided by the free space between the individual retaining elements 12. It is also possible to design the retaining elements 12 in such a way that they overlap and move relative to one another in the circumferential direction such that a collision of the edges of the retaining elements is avoided during the compression.

The material of the retaining elements 12 can be such that they serve as X-ray markers. In this way, the retaining elements 12 perform a dual function, that is to say, on the one hand, the mechanical locking of the stent on the guide wire 18 and, on the other hand, the detectability of the stent during implantation.

The body 10 forms a hollow cylindrical wall, which is formed, for example, as a closed-cell stent design. An open-cell lattice structure is likewise possible. The lattice structure can be produced by laser cutting, for example. Other types of production, for example by sputtering in conjunction with a lithography method or by a laser removal method, are likewise possible.

The connecting segment 13 forms an elastic connection between the retaining element 12 and the body 10. The elastic connection has the effect that the retaining elements 12 can move radially inward relative to the body 10 when an axial movement of the body 10 applies a radially inwardly acting force to the retaining elements 12. The elastic connection generates a restoring force, which moves the retaining elements 12 back to the opened-out position after the stent is withdrawn from the insertion system 15. In the opened-out, expanded position of the retaining elements 12, the latter extend in the longitudinal direction of the stent and thus do not protrude into the stent lumen.

The connecting segment 13 is adapted such that the retaining elements 12 and the body 10 are decoupled. For this purpose, the connecting segment can have a smaller transverse extent, i.e. a smaller extent transverse to the longitudinal axis of the stent, than the transverse extent of a retaining element 12. Other possible ways of decoupling are conceivable, for example by a suitable design of the lattice structure of the body 10.

The decoupling has the effect that the retaining elements 12 in the compressed state are less strongly curved or have a greater radius of curvature than the body 10. This means that the reduction of the stent diameter caused by the crimping into the insertion system does not have an effect on the curvature or generally on the geometry of the retaining elements 12. The curvature of the retaining elements 12 is therefore independent of the curvature of the body 10 in the compressed state. The different curvature of the retaining elements 12 and of the compressed body 10 can be seen clearly in FIGS. 1b, 1c and also in FIG. 2b.

The curvature of the retaining elements 12 is in the circumferential direction of the body 10. The curvature or the radius of curvature of the retaining element 12 is constant in the different states (compressed, expanded) that the body 10 adopts. As can be seen in FIG. 3, the retaining elements 12 arranged opposite each other are curved in the opposite direction. The retaining elements 12 are each curved concavely and follow the direction of curvature of the body 10.

A further property of the retaining elements 12 is that their contour, in the compressed state of the body 10, protrudes beyond the outer circumference thereof and thus beyond the internal diameter of the insertion system 15. This feature of the stent can be ascertained from the fact that the stent, as shown in FIG. 1a, is crimped in the area of the body and the retaining elements 12 are arranged outside the insertion system 15. Because of the decoupling of the retaining elements 12 from the body 10, they retain their original curvature. With a sufficiently large circumferential extent of the retaining elements 12, the latter protrude so far beyond the outer circumference of the body 10 or beyond the inner radius of the insertion system 15 that the retaining elements 12 ensure that, during an axial movement of the body 10, they can be moved or pressed radially inward relative to the body into the locking position by the inner wall 16 of the insertion system 15. The position of the retaining elements 12 can be seen clearly in FIGS. 1b and 1c, which each show that, because of the different radius of curvature of the retaining elements 12 and of the body 10 and because of the extent of the retaining elements 12 in the circumferential direction, they protrude beyond the outer contour of the body 10 when the body 10 is compressed.

The connecting segment 13 can be provided by one, two or more webs 14, for example, which connect a proximal end of the retaining elements 12 to the distal axial end 10a of the stent 10. In the illustrative embodiment according to FIG. 1a, two webs 14 are provided. The connecting segment 13 can be configured in a different way, for example by a thicker web or by another connecting means, for example a spring, when the radially elastic connection between the retaining element 12 and the body 10 is provided.

It is also possible that the connecting means or the connecting segment 13 and the retaining element 12 are formed in one piece, in which case the retaining element 12 is profiled and thus forms a connecting segment. For example, as in the plan view according to FIG. 5, the retaining element 12 could be designed with a substantially triangular profile or a triangular contour, in which case the vertex of the triangular contour is articulated on the body 10. The vertex has a width L₁, which is smaller than the length L₂ of a side of the triangle lying opposite the vertex. The triangle can be an equilateral or an isosceles triangle, for example. The retaining means 12 according to FIG. 5 can be made flat from solid material. Alternatively, the triangular contour or generally the contour of the retaining means 12 can be formed by webs, in particular by webs arranged in the shape of a triangle.

The retaining element 12 can have the form of a tongue or of a wing that narrows in the direction of the body 10.

Generally, the width of the connecting segment 13 is smaller than the width of the retaining element 12 extending transverse to the longitudinal direction of the body 10. This geometry applies to single and also to multiple connecting segments 13 which, for example, comprise several webs arranged alongside one another (FIG. 1a). In the case of multiple connecting segments 13, the width of the individual segments or webs arranged alongside one another is smaller than the width of the retaining element 12 extending transverse to the longitudinal direction of the body 10.

It is also possible to use, as connecting means or connecting segment 13, the lattice structure in the area of the axial end edge of the body 10, in which case the transition between the retaining element 12 and the lattice structure is such that a change in the radius of curvature of the body 10 during the crimping does not transfer to the radius of curvature of the retaining element 12.

The ratio between the length L₂ (see FIG. 5), or the transverse extent of the retaining element 12 with respect to the longitudinal axis of the stent, to the inner circumference U of the insertion device 15 or to the outer circumference of the compressed body 10 can be calculated as follows: L₂/U=sin(180°/n)/π, where n is the number of retaining elements. For four retaining elements 12 or eyelets, this gives a value of L₂/U=22%, for three eyelets a value of L₂/U=27%, and for two eyelets a value of L₂/U=32%. These values form upper limits for a corresponding number of retaining elements 12. It is possible to make the retaining elements 12 narrower, such that lower values can be achieved for the ratio L₂/U. The lower limit for the transverse extent L₂ derives from the fact that the transverse profile of a retaining element 12 protrudes beyond the outer circumference of the compressed body in the rest state. This has the effect that, by means of an axial introduction movement of the body 10 into the insertion system 15, a radial force acting inwardly on the retaining element 12 is generated, which moves the retaining element 12 into the locking position.

The maximum transverse extent L₂ is determined by the diameter of the insertion system 15, such that an insertion of the retaining elements 12 into the insertion system 15 is possible.

As can be seen in FIG. 1a, the distance L₃ (see FIG. 4) between the body 10 and the retaining element 12 is greater than the length L₂ of the retaining element 12 in the transverse direction.

This has the effect that great freedom of movement is made available to the retaining element 12 for the radial movement. It is also possible that the distance L₃ between the body 10 and the retaining element 12 is very small, such that the retaining element 12 is arranged practically on the body 10. The decoupling of the body 10 and of the retaining element 12 for the purpose of maintaining a constant radius of curvature of the retaining element 12 is achieved by a suitable transition between the retaining element 12 and the axial edge of the body 10, for example by a flexible lattice structure in the axial edge area of the body 10.

The mode of operation of the stent or generally of the medical device for releasing in a hollow organ in connection with the insertion system is illustrated in FIGS. 2a, 2b, 2c and also in FIGS. 3 to 6.

The insertion system can comprise, for example, an insertion catheter or a microcatheter and is not subject to any particular restrictions. In the system illustrated, it is a tube segment of a microcatheter for example.

As can be seen by comparing the views according to FIG. 1a and FIG. 2a, the position of the retaining elements 12 is altered by an axial relative movement between the insertion system 15 and the body 10 or the retaining elements 12. To be exact, the retaining elements 12 are moved radially inward into a locking position by the inner wall 16 of the insertion system 15. In the locking position, the retaining elements 12 cooperate with an element 18 arranged in the insertion system 15, for example with a guide wire 19 for fixing the body 10. The movement of the flexible retaining elements 12 is achieved by the fact that they have a different curvature than the crimped body 10 located in the insertion system 15. The retaining elements 12 touch the inner wall 16 at at least one location 17, in particular at two locations 17, as can be seen in FIG. 2b. In this way, the retaining elements 12 deflect radially inward and are moved into the locking position. The different positioning of the retaining elements 12 can also be seen from FIGS. 1b and 2b, which each show a view of the system according to FIGS. 1a and 2a as seen in the distal direction. FIG. 2b shows that the retaining elements 12 are arranged radially farther inward than in the view according to FIG. 1b and engage behind a projection 20 of the guide wire 19, such that the body 10 is secured at least in an axial direction on the guide wire 19. In the present illustrative embodiment, the projection 20 is designed as an annular shoulder that is arranged coaxially with respect to the guide wire 19. It is possible for the guide wire 19 to have other geometries that serve as a limit stop for the radially inwardly moved retaining elements 12.

The circular shape of the retaining elements 12 facilitates the insertion of the retaining elements 12 into the insertion system 15, since the outer edge of the insertion system 15 can slide on the curved edge of the retaining elements 12. Other profiles of the retaining elements 12 are possible, in particular profiles with a curved shape in the proximal area of the retaining elements 12. In the illustrative embodiment according to FIGS. 1a and 2a, the retaining elements 12 form plates that are movable flexibly in the radial direction. For the connection of the retaining elements 12 or plates, it is expedient to use one or more webs 14 that connect the retaining elements 12 or plates to the body 10. As is shown in FIGS. 1a and 2a, the plates, at least in the proximal or distal area, or on the side directed toward the insertion system, have a curved outer edge facilitating the insertion of the plates into the microcatheter.

In the illustrative embodiment according to FIG. 2a, the retaining elements 12 touch the inner wall 16 at two locations 17. It is also possible to modify the profile of the retaining elements 12, such that they touch the inner wall 16 only at one location or at more than two locations, for example at four, six or eight locations, and thereby to act on the radial movement of the retaining elements 12. The different locations at which the inner wall of the insertion system 15 touches the outer edge of the retaining element 12 can be arranged in succession in the longitudinal direction of the body 12. It is also possible to design the outer edge of the retaining element 12 such that there is a linear contact between the retaining element 12 and the inner wall 16. The retaining element 12 is adapted in such a way that the contact edge of the retaining element 12 extends parallel to the inner wall 16. In the case of two contact edges, these each extend parallel to the inner wall 16.

FIGS. 3 to 6 show another illustrative embodiment of the invention, in which the locking means 11 has two retaining elements 12. The different lengths, diameters and angles of the locking system in connection with the insertion system are also described in FIGS. 3 to 6.

It is clear from FIG. 3b that the length L₂ of a retaining element 12 corresponds to the maximum distance between the two outer edges of the retaining element 12. As is shown in FIG. 3b, the retaining elements 12 are moved radially inward until the distance L₂ corresponds to the internal diameter of the inner wall 16, with the retaining element 12 touching the inner wall 16 at two locations 17. It is thus possible to establish from the width of the retaining elements 12 how far the retaining elements 12 are moved radially inward. It is simply necessary to satisfy the condition that the retaining elements 12 are wider than the body 10 in the crimped state.

FIG. 3a shows that the width L₁ of the connecting segment 13 on the body 10 is smaller than the width L₂ of a retaining element. In this way, the point of articulation of the wing on the body 10 is narrower than the area of the wing away from the body, thereby producing, on the one hand, the decoupling of the body and of the retaining element 12 for generating different curvatures and, on the other hand, the flexible connection between the body 10 and the retaining element 12.

FIG. 4 shows that the connecting segments 13 or the webs 14 deform such that an angle W₁ is obtained along the length L₃ of the connecting segment. The diameter D₂ between the retaining elements 12 (see also FIG. 3b) is smaller than the diameter D₁ of the body 10. The diameter D₁ of the body 10 is achieved by compression of the body 10. The diameter D₁ of the body 10 thus corresponds substantially to the lumen of the microcatheter. The diameter D₂ derives from the diameter of a circle inscribed between the curved retaining elements 12, as is shown in FIGS. 3b and 4. The diameter D₂ thus corresponds to the maximum distance of the inner faces of the retaining elements 12 curved in opposite directions, in particular of the two retaining elements 12 curved in opposite directions.

The ratio D₁/D₂ between the diameter D₁ and the diameter D₂ comprises the following ranges:

D₁/D₂≧1.2 (lower limit)

D₁/D₂≦10 (upper limit)

The lower limit can be varied as follows:

D₁/D₂≧1.4, in particular ≧1.6, in particular ≧1.8, in particular ≧2, in particular ≧2.2, in particular ≧2.4, in particular ≧2.6, in particular ≧2.8, in particular ≧3, in particular ≧3.2, in particular ≧3.4, in particular ≧3.6, in particular ≧3.8, in particular ≧4, in particular ≧4.2, in particular ≧4.4, in particular ≧4.6, in particular ≧4.8, in particular ≧5, in particular ≧5.2, in particular ≧5.4, in particular ≧5.6, in particular ≧5.8, in particular ≧6, in particular ≧6.2, in particular ≧6.4, in particular ≧6.6, in particular ≧6.8, in particular ≧7, in particular ≧7.2, in particular ≧7.4, in particular ≧7.6, in particular ≧7.8, in particular ≧8, in particular ≧8.2, in particular ≧8.4, in particular ≧8.6, in particular ≧8.8, in particular ≧9, in particular ≧9.2, in particular ≧9.4, in particular ≧9.6, in particular ≧9.8.

The upper limit can be varied as follows:

D₁/D₂≦9.8, in particular ≦9.8, in particular ≦9.6, in particular ≦9.4, in particular ≦9.2, in particular ≦9, in particular ≦8.8, in particular ≦8.6, in particular ≦8.4, in particular ≦8.2, in particular ≦8, in particular ≦7.8, in particular ≦7.6, in particular ≦7.4, in particular ≦7.2, in particular ≦7, in particular ≦6.8, in particular ≦6.6, in particular ≦6.4, in particular ≦6.2, in particular ≦6, in particular ≦5.8, in particular ≦5.6, in particular ≦5.4, in particular ≦5.2, in particular ≦5, in particular ≦4.8, in particular ≦4.6, in particular ≦4.4, in particular ≦4.2, in particular ≦4, in particular ≦3.8, in particular ≦3.6, in particular ≦3.4, in particular ≦3.2, in particular ≦3.

The lower and upper limits mentioned above can be combined with one another to form upwardly and downwardly limited ranges.

An example of the greater diameter D₁ is D₁=0.7 mm. D₁ can vary in the range of 0.7-0.1 mm, where all intermediate values in the abovementioned range can be used for the diameter D₁.

When the body 10 or the tube segment is pushed out, the angle change W₁ is canceled again, and the retaining elements 12 return to the initial state. Thus, the state shown in FIG. 1a is resumed. The retaining elements 12 thus bear on the vessel wall and permit an unimpeded flow of blood.

FIG. 5 shows the different width of the retaining elements 12 at the end remote from the body (L₂) and at the end near the body (L₁).

FIG. 6 shows a cross section through an insertion system with a guide wire 19, which has a projection 20 or a shoulder that the retaining elements 12 engage behind in the locked position. It is also possible for the increase in diameter of the guide wire 19 to be formed by a coil that is fixed in the longitudinal direction. If a core or a projection 20 with a diameter D₃, which is greater than the diameter D₄, is moved in the proximal direction indicated by the arrow, the tube or stent segment is pushed back by the limit stop, which cooperates with the wing arranged at the angle W₁ or with the undercut obtained thereby. By means of a further limit stop (not shown), which is mounted (not shown) distally on the core or guide wire, the tube segment can also be moved in the distal direction. The diameter D₃ of the core or of the projection 20 is slightly smaller than the diameter D₁ and greater than the diameter D₂ according to FIGS. 3b and 4. In this way, the desired undercut for fixing the body 10 is obtained. The diameter D4₁ corresponds substantially to the diameter D₂ between the narrowed retaining elements or is slightly greater than the diameter D₂. The arrangement of two limit stops, spaced apart from each other in such a way that the retaining elements 12 can engage in the free space between the two limit stops, is suitable for the arrangement of the locking means 11 at the proximal axial end of the stent. In this way, the stent or generally the tube segment can be moved in both axial directions, that is to say in the proximal direction and distal direction, as a result of which the ability to position the stent or the functional element is improved. In the locked state, the functional element can be drawn back in the proximal direction into the insertion system 15 (recapturing). Moreover, by actuation of the guide wire in the distal direction, the functional element can be pushed out of the insertion system.

In summary, in this illustrative embodiment, the stent ends lie, in the rest state, on the same circumferential plane of the stent as the body 10. The radially inward bending of the retaining elements 12 is effected by the contact with the inner wall 16 of the catheter. The advantage of this is that the inwardly protruding profile of the stent during delivery is used to fix the stent in the axial direction. In the implanted state, the profile bears on the vessel wall, such that the danger of clot formation is considerably reduced.

### LIST OF REFERENCE SIGNS

10 body

10a axial end

11 locking means

12 retaining element

13 connecting segment

14 web

15 insertion system

16 inner wall

17 location

18 element

19 guide wire

20 projection

1. A medical device for releasing in a hollow organ, with a hollow cylindrical body that can be converted to a compressed state having a reduced diameter and to an expanded state having an enlarged diameter, the body having a locking means at its proximal and/or distal axial end, said locking means comprising at least one retaining element connected to the body by a connecting segment, wherein the retaining element and the body are decoupled by the connecting segment in such a way that the retaining element has a smaller curvature in the compressed state than the body, and the connecting segment elastically connects the retaining element and the body, wherein the contour of the retaining element in the compressed state of the body protrudes beyond the outer circumference of the body in such a way that the retaining element can be moved radially inward relative to the body into a locking position by the inner wall of an insertion system.

2. The device as claimed in claim 1, wherein one to four retaining elements are distributed on the circumference.

3. The device as claimed in claim 2, wherein the retaining elements are spaced apart on the circumference in the compressed state of the body.

4. The device as claimed in claim 1, wherein, in the compressed state of the body, the extent L2 of a retaining element transverse to the longitudinal direction of the body measures at least 5% of the total outer circumference of the body, in particular at least 7%, at least 10%, at least 12%, at least 14%, at least 16%, at least 18%, at least 20%, at least 22%, at least 24%, at least 26%, at least 28%, at least 30%, of the total outer circumference of the body.

5. The device as claimed in claim 1, wherein, in the compressed state of the body, the extent L2 of a retaining element transverse to the longitudinal direction of the body measures at most 32% of the total outer circumference of the body, in particular at most 30%, at most 28%, at most 26%, at most 24%, at most 22%, at most 20%, of the total outer circumference of the body.

6. The device as claimed in claim 1, wherein a retaining element is designed with a circumferential edge curved in places, in particular in a substantially circular shape.

7. The device as claimed in claim 1, wherein the retaining element comprises an X-ray marker.

8. The device as claimed in claim 1, wherein the connecting segment comprises at least one web arranged in the longitudinal direction of the body.

9. An insertion system for medical appliances having a device for releasing in a hollow organ, with a hollow cylindrical body that can be converted to a compressed state having a reduced diameter and to an expanded state having an enlarged diameter, the body having a locking means at its proximal and/or distal axial end, said locking means comprising at least one retaining element connected elastically to the body by a connecting segment, wherein the retaining element and the body are decoupled by the connecting segment in such a way that the retaining element has a smaller curvature in the compressed state than the body, and the contour of the retaining element touches the inner wall of the insertion system at at least one location, in such a way that that the retaining element is moved radially inward relative to the body into a locking position and cooperates with an element that is arranged in the insertion system and that fixes the body.

10. The insertion system as claimed in claim 9, wherein the element arranged in the insertion system comprises a guide wire having at least one projection, wherein the retaining element engages behind the projection in the locking position.

11. The insertion system as claimed in claim 10, wherein the guide wire has at least two projections, between which the retaining element engages in the locking position.
